(19) United States
(12) Patent Application Publication
(10) Pub. No.: US 2025/0339216 A1
(43) Pub. Date: Nov. 6, 2025

(54) ROBOT-ASSISTED OSTEOCHONDRAL ARTHROPLASTY

(71) Applicants: Smith & Nephew, Inc., Memphis, TN (US); Smith & Nephew Asia Pacific Pte. Limited, Singapore (SG)

(72) Inventors: Darren J. Wilson, Hull (GB); Samuel C. Dumpe, Sewickley, PA (US); Geoffrey I. Karasic, Raynham, MA (US)

(73) Assignees: Smith & Nephew, Inc., Memphis, TN (US); Smith & Nephew Asia Pacific Pte. Limited, Singapore (SG)

(21) Appl. No.: 19/184,509

(22) Filed: Apr. 21, 2025

Related U.S. Application Data
(60) Provisional application No. 63/641,443, filed on May 2, 2024.

(51) Int. Cl. A61B 34/30; A61B 34/00; A61B 34/10; A61B 34/20; A61F 2/46

(52) U.S. Cl. CPC A61B 34/30; A61F 2/4618; A61B 2034/102; A61B 2034/2065; A61B 2034/252; A61F 2002/4633

(57) ABSTRACT

A system for performing an osteochondral grafting procedure includes one or more processing devices configured to execute instructions to cause the one or more processing devices to obtain first digital data of an anatomical site of a patient, the anatomical site including a recipient site for an osteochondral graft, the recipient site including an osteochondral defect, determine, based on the first digital data, first characteristics of the recipient site, obtain second digital data of a donor site, determine, based on the second digital data, second characteristics of the donor site, and generate, based on the first and second characteristics, a virtual plug corresponding to the osteochondral graft. The virtual plug is configured to conform to the recipient site, and generating the virtual plug includes at least one of storing data defining the virtual plug and providing, on a display, visual guidance based on the virtual plug.

### CROSS-REFERENCE TO RELATED APPLICATIONS

This application claims the benefit of U.S. Provisional App. 63/641,443 filed May 2, 2024, the entire contents of which are incorporated herein by reference.

### FIELD

The present disclosure relates to robot-assisted systems and methods for performing osteochondral transplantation, including the planning and harvesting of osteochondral plugs.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Osteochondral transplantation involves harvesting tissue from donor sites (allografts or autografts) to reconstruct damaged articular cartilage in a joint such as a knee. Typically, osteochondral transplantation is a freehand technique that involves planning, graft harvesting, and graft implantation steps, which may use either open or mini-arthrotomy. In the planning step, the diameter and number of grafts to be used is determined in accordance with the geometry of a defect being repaired. In the graft harvesting step, a graft is harvested from a donor site. In the graft implantation step, the donor tissue is grafted into the recipient site.

### SUMMARY

A system for performing an osteochondral grafting procedure includes memory storing instructions and one or more processing devices configured to execute the instructions. Executing the instructions causes the one or more processing devices to obtain first digital data of an anatomical site of a patient, the anatomical site including a recipient site for an osteochondral graft, and the recipient site including an osteochondral defect, determine, based on the first digital data, first characteristics of the recipient site, obtain second digital data of a donor site, determine, based on the second digital data, second characteristics of the donor site, and generate, based on the first characteristics and the second characteristics, at least one virtual plug corresponding to the osteochondral graft. The at least one virtual plug is configured to conform to the recipient site, and generating the at least one virtual plug includes at least one of storing data defining the at least one virtual plug and providing, on a display, visual guidance based on the at least one virtual plug.

In other features, the osteochondral grafting procedure includes at least one of an osteochondral autograft transplantation (OAT) procedure and an osteochondral allograft (OCA) procedure. Obtaining the first digital data includes generating a surface model of the anatomical site and performing segmentation of the recipient site based on the surface model, obtaining the second digital data includes obtaining a digital template of the donor site, and generating the at least one virtual plug includes performing a matching process to obtain anatomic shapes and curvatures of both the recipient site and the donor site and generating the at least one virtual plug based on results of the matching process. Obtaining the digital template includes digitally segmenting the donor site into a plurality of donor sites. Performing the matching process includes comparing shapes and curvatures of the recipient site to shapes and curvatures of the plurality of donor sites. Performing the matching process includes generating the at least one virtual plug based on one of the plurality of donor sites and superimposing the at least one virtual plug on the recipient site.

In other features, generating the at least one virtual plug includes determining a topographical match between the recipient site and the donor site based on the first and second characteristics. Generating the at least one virtual plug includes at least one of translating and rotating the at least one virtual plug to assess conformity of the at least one virtual plug to the recipient site. Assessing the conformity includes calculating an average error of a topographic mismatch between the at least one virtual plug and the recipient site. Assessing the conformity includes calculating a least squares distance error between respective surfaces of the at least one virtual plug and the recipient site. The first characteristics include first radius of curvature data for the recipient site and the second characteristics include second radius of curvature data for the donor site, and generating the at least one virtual plug includes generating a best-fit sphere based on the first radius of curvature data and the second radius of curvature data.

In other features, Generating the at least one virtual plug includes identifying respective diameters, heights, and surface slopes of a plurality of virtual plugs based on the first and second characteristics. Generating the at least one virtual plug includes executing a shape-packing algorithm to select a plurality of virtual plugs. Executing the instructions further causes the one or more processing devices to control a robot to at least one of obtain, based on the at least one virtual plug, the osteochondral graft from the donor site and implant the osteochondral graft at the recipient site.

A method for performing an osteochondral grafting procedure includes, using one or more processing devices, obtaining first digital data of an anatomical site of a patient, the anatomical site including a recipient site for an osteochondral graft, and the recipient site including an osteochondral defect, determining, based on the first digital data, first characteristics of the recipient site, obtaining second digital data of a donor site, determining, based on the second digital data, second characteristics of the donor site, and generating, based on the first characteristics and the second characteristics, at least one virtual plug corresponding to the osteochondral graft, the at least one virtual plug being configured to conform to the recipient site. Generating the at least one virtual plug includes at least one of storing data defining the at least one virtual plug and providing, on a display, visual guidance based on the at least one virtual plug.

In other features, the osteochondral grafting procedure includes at least one of an osteochondral autograft transplantation (OAT) procedure and an osteochondral allograft (OCA) procedure. Obtaining the first digital data includes generating a surface model of the anatomical site and performing segmentation of the recipient site based on the surface model, obtaining the second digital data includes obtaining a digital template of the donor site, and generating the at least one virtual plug includes performing a matching process to obtain anatomic shapes and curvatures of both the recipient site and the donor site and generating the at least one virtual plug based on results of the matching process. Generating the at least one virtual plug includes at least one of determining a topographical match between the recipient site and the donor site based on the first and second characteristics, at least one of translating and rotating the at least one virtual plug to assess conformity of the at least one virtual plug to the recipient site, identifying respective diameters, heights, and surface slopes of a plurality of virtual plugs based on the first and second characteristics, and executing a shape-packing algorithm to select a plurality of virtual plugs.

In other features, the first characteristics include first radius of curvature data for the recipient site and the second characteristics include second radius of curvature data for the donor site, and generating the at least one virtual plug includes generating a best-fit sphere based on the first radius of curvature data and the second radius of curvature data. The method further includes controlling a robot to at least one of obtain, based on the at least one virtual plug, the osteochondral graft from the donor site and implant the osteochondral graft at the recipient site.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

For a detailed description of example embodiments, reference will now be made to the accompanying drawings in which:

FIGS. 1A and 1B show an example surgical system in accordance with at least some embodiments;

FIG. 1C shows a conceptual drawing of an example surgical site with various objects within the surgical site tracked in accordance with at least some embodiments;

FIGS. 2A and 2B illustrate construction of a model of a femur in accordance with at least some embodiments;

FIG. 3A shows a model of an example inferior aspect of a right digitized femur in accordance with at least some embodiments;

FIG. 3B shows example digital templating of donor sites on an inferior aspect of a right digitized femur in accordance with at least some embodiments;

FIGS. 4A and 4B show example generation of a best fit sphere in accordance with at least some embodiments;

FIGS. 5A, 5B, and 5C show example generation of virtual plugs in accordance with at least some embodiments;

FIGS. 6A, 6B, 6C, and 6D show example plug assemblies or patterns in accordance with at least some embodiments;

FIGS. 7A, 7B, and 7C show example visual guidance for harvesting of plugs in accordance with at least some embodiments;

FIG. 8 shows an example method for performing osteochondral arthroplasty techniques for an OAT procedure in accordance with at least some embodiments;

FIG. 9 shows an example method for performing osteochondral arthroplasty techniques for an OCA in accordance with at least some embodiments; and

FIG. 10 shows an example computer system or computing device configured to implement the various systems and methods of the present disclosure.

In the drawings, reference numbers may be reused to identify similar and/or identical elements.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

Similarly, spatial and functional relationships between elements (for example, between device, modules, circuit elements, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. Nevertheless, this paragraph shall serve as antecedent basis in the claims for referencing any electrical connection as “directly coupled” for electrical connections shown in the drawing with no intervening element(s).

Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure. As one example, numerical values that are described as “approximate” or “approximately” as used herein may refer to a value within +/−5% of the stated value.

“A”, “an”, and “the” as used herein refers to both singular and plural referents unless the context clearly dictates otherwise. By way of example, “a processor” programmed to perform various functions refers to one processor programmed to perform each and every function, or more than one processor collectively programmed to perform each of the various functions. To be clear, an initial reference to “a [referent]”, and then a later reference for antecedent basis purposes to “the [referent]”, shall not obviate the fact the recited referent may be plural.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context. As used herein, the phrase at least one of A, B, and C should be construed to mean a logical (A OR B OR C), using a non-exclusive logical OR, and should not be construed to mean “at least one of A, at least one of B, and at least one of C.”

The terms “input” and “output” when used as nouns refer to connections (e.g., electrical, software) and/or signals, and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections and/or signals transmitted or received by those connections. In systems implemented in software, these “inputs” and “outputs” define parameters read by or written by, respectively, the instructions implementing the function. In examples where used in the context of user input, “input” may refer to actions of a user, interactions with input devices or interfaces by the user, etc.

“Controller,” “module,” or “circuitry” shall mean, alone or in combination, individual circuit components, an application specific integrated circuit (ASIC), a microcontroller with controlling software, a reduced-instruction-set computer (RISC) with controlling software, a digital signal processor (DSP), a processor with controlling software, a programmable logic device (PLD), a field programmable gate array (FPGA), or a programmable system-on-a-chip (PSOC), configured to read inputs and drive outputs responsive to the inputs.

As used to describe various surgical instruments or devices, such as a probe, the term “proximal” refers to a point or direction nearest a handle of the probe (e.g., a direction opposite the probe tip). Conversely, the term “distal” refers to a point or direction nearest the probe tip (e.g., a direction opposite the handle).

For the purposes of this disclosure, a non-transitory computer readable medium (or computer-readable storage medium/media) stores computer data, which data can include computer program code (or computer-executable instructions) that is executable by a computer, in machine-readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure, the term “server” should be understood to refer to a service point that provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4th or 5th generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example. In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, photopletismograph (PPG), Doppler and transmit-time flow meter, laser Doppler, an endoscopic device neuromodulation device, a neurostimulation device, and the like, or some combination thereof.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Osteochondral transplantation involves harvesting tissue from donor sites (allografts or autografts) to reconstruct damaged articular cartilage in a joint such as a knee, often due to trauma or overuse. Typically, osteochondral transplantation is a freehand technique that involves planning, graft harvesting, and graft implantation steps, which may use either open or mini-arthrotomy. In the planning step, the diameter and number of grafts to be used is determined in accordance with the geometry of a defect being repaired. In the graft harvesting step, a graft is harvested from a donor site. In the graft implantation step, the donor tissue is grafted into the recipient site. The osteochondral transplantation process is time consuming and requires careful harvesting and implantation in order to achieve more than or equal to 80% coverage with well-integrated and stabilized grafts. Moreover, cognitive overload can occur due to the number of sequential tasks that have to be carefully planned for in order to obtain a satisfactory outcome for the patient.

In some examples, osteochondral autograft transplantation (OAT), also known as mosaicplasty, uses donor osteochondral cylinders removed from relatively low-weight bearing regions of the lateral supracondylar or the intercondylar notch area of the femur. Mosaicplasty may be indicated for treating small-sized full-thickness chondral defects, typically between 1 and 4 cm² in area. In other examples, osteochondral allograft (OCA) transplantation surgery involves taking a larger amount of tissue from a cadaver rather than from the patient's own knee.

In standard mosaicplasty procedure, 60%-70% of a defected area is filled with hyaline cartilage, and a remaining 30%-40% is filled with fibrous cartilage. In order to increase the area filled with hyaline cartilage, either smaller diameter grafts can be used or larger grafts can be overlapped, and each of these techniques has limitations. Smaller grafts may lead to instability and collapse of grafts, whereas overlapping may decrease fixation strength. In either case, as a result, early weight-bearing and active motion would be avoided.

Various imaging techniques (radiographs, MRI, CT, etc.) are used to determine the best approach for repairing the defect (e.g., predicting lesion size and shape and an ideal resection allograft and fixation construct).

Osteochondral arthroplasty systems and methods according to the present disclosure are configured to implement computer-aided and robot-assisted surgical navigation techniques to facilitate accurate planning (e.g., templating), harvesting, and implanting of osteochondral tissue plugs. For example, various computer-aided or assisted surgery (CAS) and surgical navigation systems support surgeons in planning and performing complex surgical procedures with increased precision and accuracy. In some examples, computer-assisted surgical procedures (e.g., surgical procedures associated with a knee or knee joint, a hip or hip joint, etc.) may implement either image or imageless-based robot-assisted techniques. For example, surgical navigation systems can implement robot-assisted techniques to aid surgeons in locating patient anatomical structures, guiding surgical instruments, and implanting medical devices with a high degree of accuracy as described below in more detail.

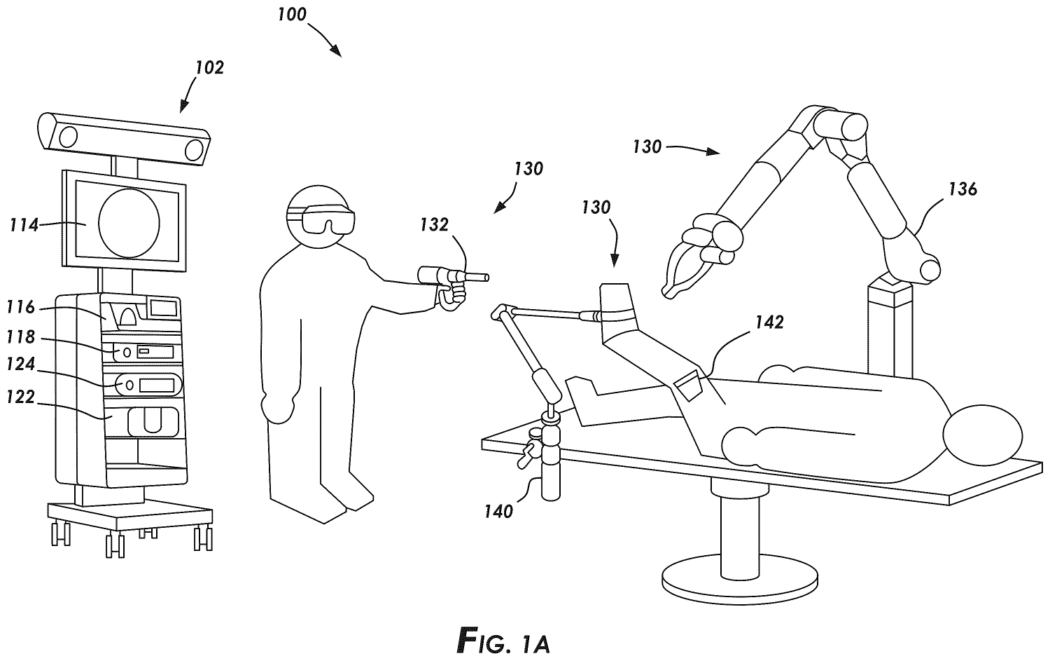

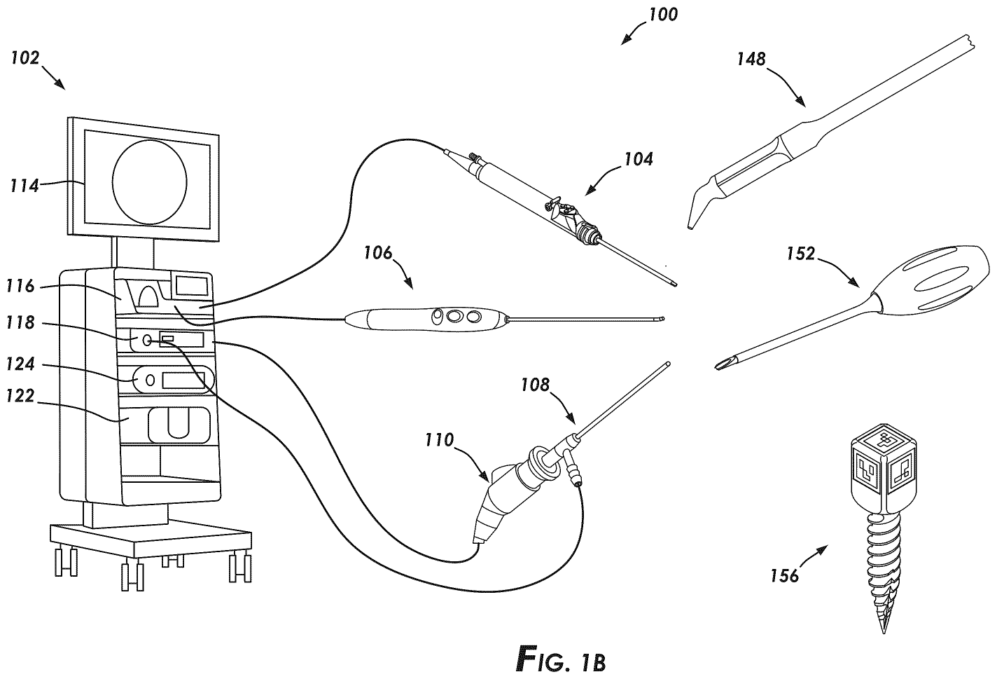

FIGS. 1A and 1B provides an illustration of an example surgical system (e.g., a computer-assisted surgical system, or CASS) 100 according to some embodiments. In some examples, the surgical system 100 may be configured to implement a video-based navigation system (e.g., an arthroscopic video-based navigation system). In some examples as described below, the surgical system 100 is configured to use various computers or computing devices, robotics, and imaging technology to aid surgeons in performing orthopedic surgery procedures, such as osteochondral arthroplasty. For example, surgical navigation systems can aid surgeons in locating patient anatomical structures, guiding surgical instruments, and implanting medical devices with a high degree of accuracy. Surgical navigation systems often employ various forms of computing technology to perform a wide variety of standard and minimally invasive surgical procedures and techniques. Moreover, these systems allow surgeons to more accurately plan, track, and navigate the placement of instruments and implants relative to the body of a patient, as well as conduct pre-operative and intra-operative body imaging. The potential benefits of surgical navigation-assisted early knee intervention include increased precision during graft harvest, enhanced accuracy during defect preparation, and guided graft placement. This, in turn, leads to improved accuracy, reproducibility, and potentially better clinical outcomes compared to freehand techniques. For example, improved accuracy of the implant and fixation device, reduced complications, such as iatrogenic injury of the surrounding tissues, faster recovery time for the patient and enhanced functional outcomes, i.e. precise repair can lead to better joint function and a faster return to activities of daily living.

The surgical system 100 may include a tower or device cart 102 and various tools and instruments, such as an example mechanical resection instrument 104, an example plasma-based ablation instrument (hereafter just ablation instrument 106), and an endoscope in the example form of an arthroscope 108 and attached camera head or camera 110. In the example systems, the arthroscope 108 may be a rigid device, unlike endoscopes for other procedures, such as upper-endoscopies. The device cart 102 may comprise a display device 114, a resection controller 116, and a camera control unit (CCU) together with an endoscopic light source and video (e.g., a VBN) controller 118. In example cases the combined CCU and video controller 118 not only provides light to the arthroscope 108 and displays images received from the camera 110, but also implements various additional aspects, such as registering a three-dimensional bone model with the bone visible in the video images, and providing computer-assisted navigation during the surgery. Thus, the combined CCU and video controller are hereafter referred to as surgical controller 118. In other cases, however, the CCU and video controller may be a separate and distinct system from the controller that handles registration and computer-assisted navigation, yet the separate devices would nevertheless be operationally coupled.

The example device cart 102 further includes a pump controller 122 (e.g., single or dual peristaltic pump). Fluidic connections of the mechanical resection instrument 104 and ablation instrument 106 to the pump controller 122 are not shown so as not to unduly complicate the figure. Similarly, fluidic connections between the pump controller 122 and the patient are not shown so as not to unduly complicate the figure. In the example system, both the mechanical resection instrument 104 and the ablation instrument 106 are coupled to the resection controller 116 (e.g., a dual-function controller). In other cases, however, there may be a mechanical resection controller separate and distinct from an ablation controller. The example devices and controllers associated with the device cart 102 are merely examples, and other examples include vacuum pumps, patient-positioning systems, robotic arms holding various instruments, ultrasonic cutting devices and related controllers, patient-positioning controllers, and robotic surgical systems. In some examples, the device cart 102 may include one or more controllers (e.g., a robot controller 124) configured to control components of a robotic surgical system, such as one or more robots, tools or instruments associated with the one or more robots, etc.

In some examples, the surgical system 100 may include or implement an effector platform 130 configured to position surgical tools relative to a patient during surgical procedures. The exact components of the effector platform 130 will vary, depending on the embodiment employed. For example, for a knee surgery, the effector platform 130 may include an end effector 132 that holds surgical tools or instruments during use. The end effector 132 may be a handheld device or instrument used by the surgeon or, alternatively, the end effector 132 can include a device or instrument held or positioned by a robot (e.g., a robotic arm) 136. While shown having only one robotic arm, the robot 136 may include multiple arms or other components. For example, the robot 136 may include one or more robotic arms on respective sides of an operating table, two devices on one side of the operating table, etc. The robot 136 may be mounted directly to the table, located next to the table on a floor platform (not shown), mounted on a floor-to-ceiling pole, mounted on a wall or ceiling of an operating room, etc. The floor platform may be fixed or moveable.

The effector platform 130 may include a limb positioner 140 for positioning patient limbs during surgery. The limb positioner 140 may be operated manually by the surgeon or alternatively change limb positions based on instructions received from a computing device (e.g., a controller of the surgical system 100). While one limb positioner 140 is shown in FIG. 1A, in some embodiments there may be multiple devices. For example, there may be one limb positioner 140 on each side of the operating table, two devices on one side of the table, etc. The limb positioner 140 may be mounted directly to the table, located next to the table on a floor platform (not shown), mounted on a pole, or mounted on a wall or ceiling of an operating room. The limb positioner 140 may include, as examples, an ankle boot, a soft tissue clamp, a bone clamp, or a soft-tissue retractor spoon, such as a hooked, curved, or angled blade. In some embodiments, the limb positioner 140 may include a suture holder to assist in closing wounds.

The effector platform 130 may include tools, such as a screwdriver, light or laser, to indicate an axis or plane, bubble level, pin driver, pin puller, plane checker, pointer, finger, or some combination thereof.

Resection equipment (such as the mechanical resection instrument 104) can be used to perform bone or tissue resection using, for example, mechanical, ultrasonic, or laser techniques. Examples of resection equipment include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the resection equipment is held and operated by the surgeon during surgery. In other embodiments, the effector platform 130, robot 136, etc. may be used to hold the resection equipment during use.

The effector platform 130 also can include a cutting guide or jig 142 that is used to guide saws or drills used to resect tissue during surgery. Such cutting guides 142 can be formed integrally as part of the effector platform 130 or robot 136, or cutting guides can be separate structures that can be matingly and/or removably attached to the effector platform 130 or the robot 136. The effector platform 130 or robot 136 can be controlled by the surgical system 100 to position a cutting guide or jig adjacent to the patient's anatomy in accordance with a pre-operatively or intraoperatively developed surgical plan such that the cutting guide or jig will produce a precise bone cut in accordance with the surgical plan.

Various tracking systems use one or more sensors to collect real-time position data to locate patient anatomy and surgical instruments. For example, a tracking system may provide a location and orientation of the end effector 132 during the procedure. In addition to positional data, data from the tracking system can be used to infer velocity/acceleration of anatomy/instrumentation, which can be used for tool control. In some embodiments, the tracking system may use a fiducial marker, tracker array, etc. attached to the end effector 132 to determine the location and orientation of the end effector 132. The position of the end effector 132 may be inferred based on the position and orientation of the tracking system and a known relationship in three-dimensional space between the tracking system and the end effector 132. Various types of tracking systems may be used in various embodiments of the present disclosure including, without limitation, Infrared (IR) tracking systems, electromagnetic (EM) tracking systems, video or image based tracking systems, and ultrasound registration and tracking systems. Using the data provided by the tracking system, the surgical system 100 can detect objects and prevent collision. For example, the surgical system 100 can prevent the robot 136 and/or the end effector 132 from colliding with soft tissue.

Any suitable tracking system can be used for tracking surgical objects and patient anatomy. For example, a combination of IR and visible light cameras can be used in an array. Various illumination sources, such as an IR LED light source, can illuminate the scene allowing three-dimensional imaging to occur. In some embodiments, this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. Further, some imaging devices may be of suitable resolution or have a suitable perspective on the scene to pick up information stored in quick response (QR) codes or barcodes. In some embodiments, a camera may be mounted on the robot 136.

FIGS. 1B and 1C further show additional instruments that may be present during a surgical procedure, including instruments associated with various tracking and registration techniques. In particular, an example probe 148 (e.g., shown as a touch probe, but which may be a touchless probe in other examples), a drill guide or aimer 152, and a fiducial marker (e.g., a bone fiducial, fiducial markers located on instruments, robotic components, etc.) 156 are shown. The probe 148 may be used during the surgical procedure to provide information to the surgical controller 118, such as information to register a three-dimensional bone model to an underlying bone visible in images captured by the arthroscope 108 and camera head 110. In some surgical procedures, the aimer 152 may be used as a guide for placement and drilling with a drill wire to create an initial or pilot tunnel through the bone. The fiducial marker 156 may be affixed or rigidly attached to the bone and serve as an anchor location for the surgical controller 118 to know the position and orientation of the bone (e.g., after registration of a three-dimensional bone model). Additional tools and instruments may be present, such as the drill wire, various reamers for creating the throughbore and counterbore aspects of a tunnel through the bone, and various tools, such as for suturing and anchoring a graft. These additional tools and instruments are not shown so as not to further complicate the figure. In some examples, the surgical system 100 may be configured to implement registration and/or tracking techniques using one or more of the fiducial markers 156, such as techniques described in more detail in U.S. patent application Ser. No. 19/170,202, filed on Apr. 4, 2024, the entire contents of which are incorporated herein by reference.

All or portions of an example procedure according to the principles of the present disclosure can be conducted arthroscopically. An example procedure is computer-assisted in the sense that the surgical system 100 is used for control of the robot 136, navigation (arthroscopic or otherwise) within the surgical site, etc. More particularly, in example systems, the surgical system 100 can provide computer-assisted navigation during the procedure by tracking locations of various objects within the surgical site, such as the location of the bone within the three-dimensional coordinate space of the view of a camera (e.g., an arthroscope), and location of the various instruments within the three-dimensional coordinate space of the view of the camera.

The surgical system 100 may be configured to implement and/or facilitate osteochondral arthroplasty techniques (e.g., OCA and OATS (mosaicplasty)) according to the present disclosure. The techniques described herein may include one or more pre-operative or planning phases, stages, or steps followed by one or more intraoperative phases, stages, or steps. For example, an osteochondral arthroplasty surgical procedure may begin with a planning phase. Planning for an example imaged-based robotic assisted procedure may start with imaging (e.g., X-ray imaging, computed tomography (CT), magnetic resonance imaging (MRI)) of the anatomy of the patient, including the relevant anatomy (e.g., for a knee procedure the lower portion of the femur, the upper portion of the tibia, and the articular cartilage; for a hip procedure, an upper portion of the femur, the acetabulum/hip joint, pelvis, etc.). The imaging may be preoperative imaging, hours or days before the intraoperative repair, or the imaging may take place within the surgical setting just prior to the intraoperative repair. The discussion that follows assumes MRI imaging, but again many different types of imaging may be used. The image slices from the MRI imaging can be segmented such that a volumetric model or three-dimensional model of the anatomy is created. Any suitable currently available, or after developed, segmentation technology may be used to create the three-dimensional model to help plan the surgery and determine the best approach for repairing the defect, promoting better integration and stability of the transplanted grafts. More specifically to the example of anterior cruciate ligament repair, a three-dimensional bone model of the lower portion of the femur, including the femoral condyles, is created. Conversely, for a hip procedure, a three-dimensional model of the upper portion of the femur and at least a portion of the pelvis (e.g., the acetabulum) is created.

Using the three-dimensional bone model, an operative plan is created. For a knee procedure, the results of the planning may include: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia. Other surgical parameters may also be selected during the planning, such as tunnel throughbore diameters, tunnel counterbore diameters and depth, desired post-repair flexion, and the like (e.g., to guide placement of anchors, screws, or grafts, ensuring proper seating and fixation), but those additional surgical parameters are omitted so as not to unduly complicate the specification.

The intraoperative phase may include steps and procedures for setting up the surgical system to perform the various repairs. It is noted, however, that some of the intraoperative aspects (e.g., optical system calibration) may take place before any portals or incisions are made through the patient's skin, and in fact before the patient is transported into the surgical room. Nevertheless, such steps and procedures may be considered intraoperative as they take place in the surgical setting and with the surgical equipment and instruments used to perform the actual repair.

Planning techniques described herein may provide a surgical plan providing information that may include, but is not limited to:
a reconstruction of the original articular joint surface obtained from a statistical shape model or by matching the topography of the autograft/allograft and defect sites using either a rigid transformation between the surfaces that minimized RMS error or variance between radius of curvatures;
an evaluation of cartilage thickness in donor and receptor areas so that the thickness of the reconstructed cartilage matches the initial thickness of the cartilage to avoid degeneration at the bone/cartilage interface or excessive rim stresses around the defect perimeter;
size, shape, and location of the digitized osteochondral defect relative to the reconstructed original articular joint surface;
position and orientation of the donor sites for harvesting each osteochondral cylindrical graft and allograft tissue so that the orientation of the surface of each osteochondral graft matches with the anticipated orientation of the surface of the recipient site;
for an OAT procedure, an optimal mosaic template, such as a model, to ensure defect coverage with minimum overlapping and spacing/gapping;
for an OAT procedure, an ideal orientation of each plug matched to the predicted surface orientation at the corresponding delivery site (e.g., to aid in drilling the recipient holes and positioning the osteochondral plugs for a tight, stable fit); and
for an OAT procedure, an optimum position, depth, and perpendicularity of a harvest location of each plug and respective delivery locations (e.g., using optically tracked surgical instruments).

Either pre-operatively or intraoperatively, defects can be prepared with a level of macroscopic rough surface topography required to increase contact area and friction, which can promote allograft stability. Allograft tissue can then be optimally fixated within the defect using pins and screws (e.g., pins and screws inserted using robotics techniques). Use of non-rotationally symmetric plugs improves filling of the defect volume while minimizing impact to healthy tissue.

As one example, osteochondral arthroplasty techniques of the present disclosure include using topographic matching algorithms to compare the topography of selected donor and recipient sites to determine the site with the best topographic match for repairing defects in the weight-bearing portions of the condyles. Custom intra-operative surgical planning templates are created based upon a three-dimensional reconstruction of the specific anatomical structures of the patient. For example, in the case of a knee osteochondral defect, a 3D model of the patient's femur and a vector of points representative of the contour of the injured osteochondral defect are created. The size and elongation of the osteochondral defect is then captured by fitting a 2D ellipse to the contour of the defect, while the anatomical shape is described by fitting an elliptic or hyperbolic paraboloid to the external surface. The 3D model of the diseased bone and the osteochondral defect are subsequently captured in the same coordinate reference frame using algorithms to support digitized templating, trimming, and implantation of the graft tissues.

Once the osteochondral defect boundary has been digitized, distinct areas of both the donor and recipient sites of the lateral and medial femoral condyles (e.g., in respective models) are morphed to match respective anatomic shapes and curvatures. Planning software is used to template an exact location of the donor site tissue (e.g., from either a low-stress joint region of the patient (an autograft) or other donor tissue (an allograft)) that is used to fill the weight-bearing portion of the injured joint. In the case of osteochondral autograft transplantation, the 3D model of the digitized femur is used to select the optimum location of the donor plugs from either the medial or lateral trochlea ridge and medial intercondylar notch. Selecting the optimum location can be achieved by comparing the radius of curvature (RoC) of the three segmented donor sites for harvesting the plugs with the recipient site and evaluating any differences that may exist between them. The planning software can also be used to select the size (e.g., diameter and depth), number, and angulation of the plugs harvested from the recipient site to ensure that the plugs are congruent and not too proud (i.e., projecting) or counter-sunk with respect to the surrounding tissue. A customized reference tracker (e.g., a fiducial marker) is fixed to both the recipient and donor harvesting guides, referencing the exact position in relation to the predefined articular surface. Consequently, the permanent position and depth of the guide tip in relation to the articular surface is continuously visualized in real-time.

In some examples, robot-assisted mini-arthrotomy (e.g., for defects up to 4 cm long) according to the present disclosure can support enhanced templating of the injured cartilage region through precise knowledge of the size, depth, and location of the osteochondral defect and the recipient sites in the low-weight bearing region of the joint. Robotically-tracked harvesting guides and cutting tools would also prevent over drilling or misalignment of the holes in the defect to seat the harvested plugs supporting the precise restoration of the curvature and congruency of the repair, which is technically demanding and difficult to achieve manually. In turn, this would lead to a more simplified digital workflow and durable repair of the articular defect attributed to a more favorable biomechanical environment. Pre-calibrated tracked tools would allow for greater accuracy and a reduction in the time needed for registration.

A robotic-controlled procedure may also improve the vertical positioning of the grafts with regards to the joint surface and the congruity of the graft material in relation to the surrounding articular surface when compared with a freehand technique. Further, templating and preparation of the healthy donor grafts during allograft procedures can be enhanced, ensuring they are the correct size and shape.

Although described herein with respect to, generally, osteochondral arthroplasty, the principles of the present disclosure can be used in other types of surgical procedures (e.g., other types of concomitant procedures such as ACL reconstruction or high tibial osteotomy) that may be performed in addition to osteochondral repair. Further, although described with respect to OAT and OCA procedures, the principles of the present disclosure may be applied to other type of grafting procedures, which may be referred to generally as graft or grafting procedures.

In some examples, automatic planning techniques according to the principles of the present disclosure include performing imageless femur landmark registration. For example, rigid reference markers (e.g., fiducial markers as described herein) are attached to the distal femur and proximal tibia (e.g., for a robot-assisted osteochondral knee defect repair procedure). A bone morphing algorithm is used to define the anatomical landmarks during knee flexion using a digitized tracked pointer probe for initial sizing of the patient femur. Example landmarks may include, but are not limited to, a knee center, the most posterior medial and lateral points at 90° of flexion on the apex on the condyle, and an intra-articular anterior notch point, which can be used as a reference during implant planning to prevent notching of the anterior cortex. The knee center can be referenced as part of the HKA (hip-knee-ankle) weight-bearing axis. Point collection for the knee center, along with the hip center collection, and defined tibial and femoral cutaneous landmarks, defines the femur mechanical axis, which can be used as an axis to orientate the harvested grafts.

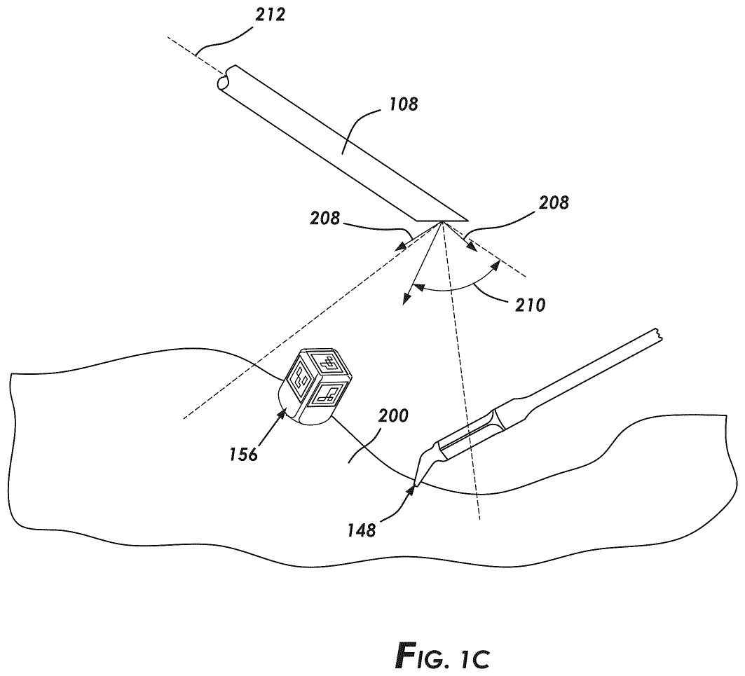

Planning techniques may include generating 3D virtual bone and focal defect models by gathering/obtaining digital data associated with a recipient site. The bone morphing algorithm is used to define the femoral intra-articular landmarks including the donor and recipient sites of the lateral and medial sites of the femoral trochlear and the lateral and medial femoral condyles. For example, accurate planning may include performing an estimation of an original articular surface (i.e., the surface prior to formation/presence of the defect). A healthy (i.e., defect-free) femur model can be constructed by means of a statistical shape model that ignores the presence of a defect and reconstructs a “missing” cartilage surface (i.e., as if the defect is not present) using cubic spline or other suitable techniques. As shown in FIG. 2A, surface points 200 can be obtained (and stored, for example as surface model data usable by and provided to software) by manually by moving a point probe over an entire surface of a femur 204. The femur 204 is shown in FIG. 2A without a defect. As another example, a 3D femur model and articular cartilage can also be obtained by segmenting pre-operative MRI or CT-arthrogram images. As shown in FIG. 2B, the size and elongation of an osteochondral defect 208 may be captured by fitting a 2D ellipse to the contour of the defect, while a corresponding anatomical shape can be described by fitting an elliptic or hyperbolic paraboloid to the external surface. A boundary of the osteochondral defect 208 can then be digitized (e.g., to determine the size and shape of the defect 208) to assist with planning and harvesting of the donor tissue. Registration can then be made between the 3D bone model of the femur and the osteochondral defect (e.g., using a combined pairpoint and surface matching algorithm).

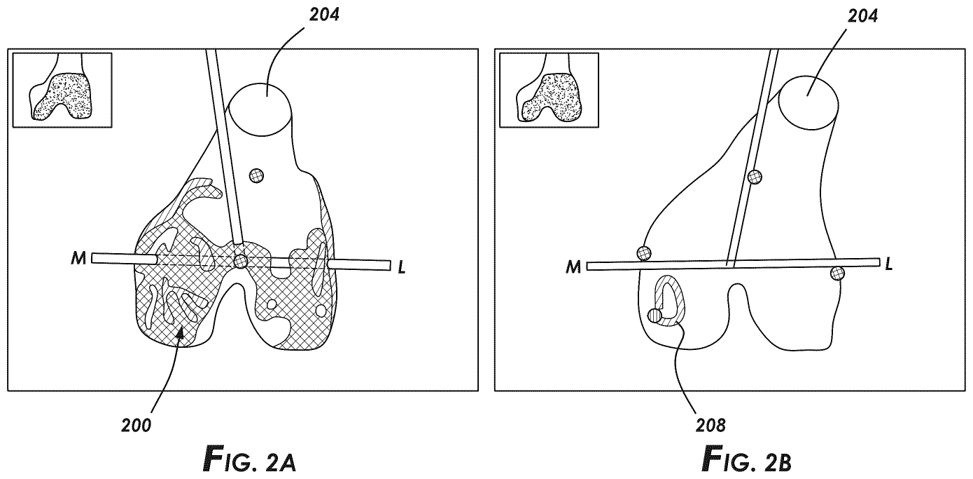

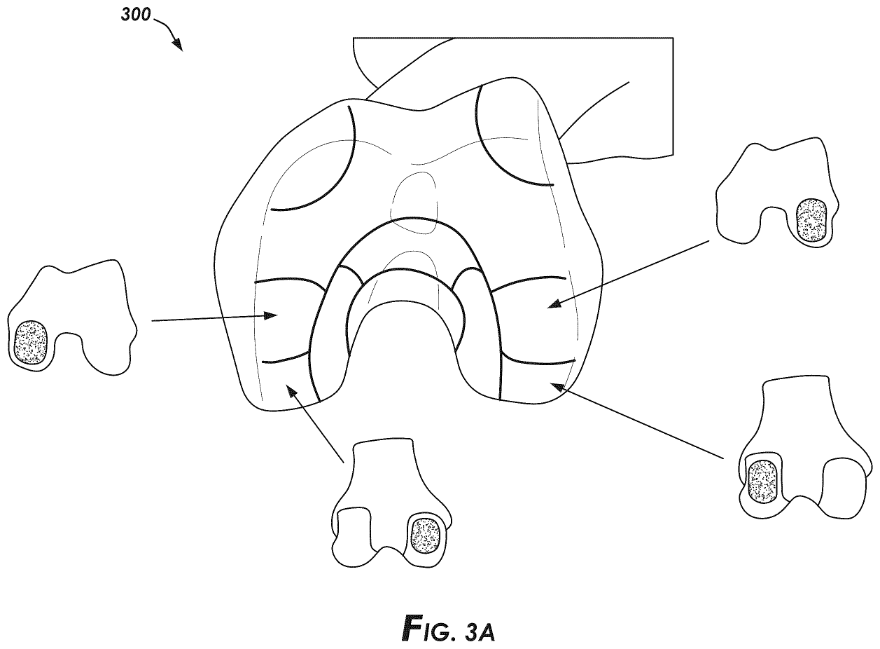

Segmentation of an osteochondral defect recipient site (e.g., a site on a femur of the patient that includes the defect 208) can then be performed using the models (e.g., model data) obtained as described above in FIGS. 2A and 2B. Once the osteochondral defect boundary has been digitized and introduced into a same coordinate reference system as the native femur model, the defect boundary may be compared to a library of existing osteochondral defect sites. FIG. 3A shows a model or other image of an example inferior aspect of a right digitized femur 300 illustrating the most common recipient sites associated with osteochondral defects. For example, existing defect sites may include, but are not limited to, a distal medial femoral condyle, a distal lateral femoral condyles, a posterior medial femoral condyle, and/or a posterior lateral femoral condyle.

FIG. 3B illustrates example digital templating of donor sites on an inferior aspect of a right digitized femur 312. As shown in FIG. 3B, typical locations for harvesting donor tissue are illustrated. Example sites include, but are not limited to, a medial trochlear ridge, a lateral intercondylar notch, a medial intercondylar notch, and a lateral trochlear ridge. As shown, pre-identified “viable” donor sites within the lateral and medial femoral condyles are divided into four regions corresponding to the lateral trochlear, medial trochlear ridges, lateral intercondylar notch, and medial intercondylar notch to assist with topographic matching. Additionally, a central intercondylar notch can be bounded/defined. Each of the four segmented zones can be further sub-divided into sub-zones or sites. As shown in FIG. 3B, each zone is sub-divided into superior (1, 4, 7, and 10), middle (2, 5, 8, and 11), and inferior (3, 6, 9, and 12) sites. In this manner, determination of optimum size (e.g., 4, 6, 8 or 10 mm diameter) and location for harvesting the plugs given the available surface area and articular cartilage thickness and matched radius of curvature with the injured/defect site can be facilitated.

In some examples, topographic and geometric matching is performed to obtain anatomic shape and curvature of both donor and recipient sites. Topographic matching between the donor and recipient sites can be achieved using various techniques. As one example, software can be configured to compare the relative curvature of any selected donor and recipient site on the articular surface of the distal femur. Each osteochondral graft can be selected from potential donor sites and then superimposed onto a selected recipient site on the weight bearing region of the femur. The graft can then be translated and rotated in space until the correct angle and orientation between the donor and recipient sites is obtained that promotes a congruent surface. Position optimization can be achieved with respect to six variables (e.g., three translation planes and three rotation planes) that are solved by a nonlinear programming equation. The software can then calculate the average error of topographic mismatch (e.g., in mm) between the donor and recipient sites by optimizing the position of the donor site with respect to the recipient site and calculating the least squares distance error between the two surfaces.

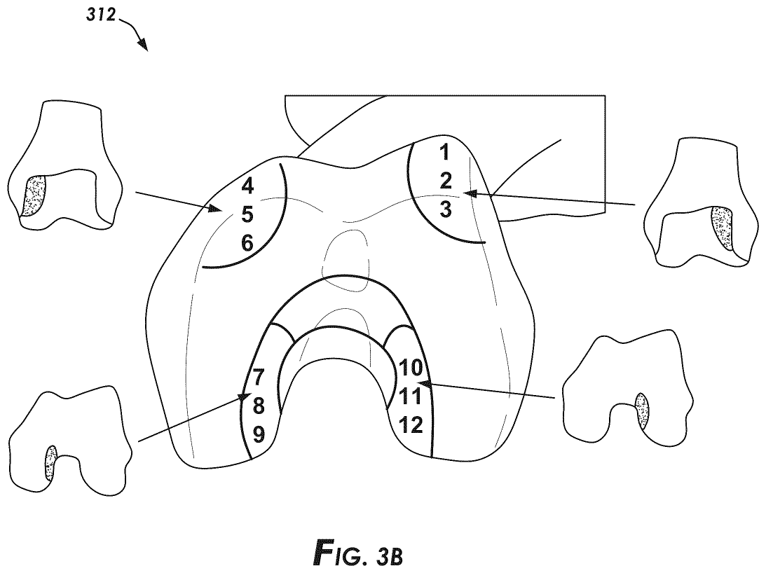

As another example, a best fit sphere can be generated for each randomly selected point of interest across donor or recipient sites as shown in FIGS. 4A and 4B. For example, FIGS. 4A and 4B show a left distal femur 400 with a “best fit” radius of curvature (RoC) sphere 404 superimposed onto an osteochondral defect site 408 located in the distal lateral femoral condyle, in semi-axial and semi-sagittal views, respectively. A mean radius of curvature (Roe) can be calculated for each donor/recipient region from a cloud of data points obtained from the digitized probe. As an example, RoC typically ranges between 5 and 12 mm with an expected variance (R²) between the recipient and donor regions being less than 1. The direction perpendicular to the plane tangential to the surface of the femur is used as the direction for simulated graft harvesting during templating. A contacted articular surface as shown at 412 represents one example simulated graft recipient site with a diameter of 6 mm. As an example, other plug sizes can be considered during the planning stage from 2 mm up to 10 mm diameter.

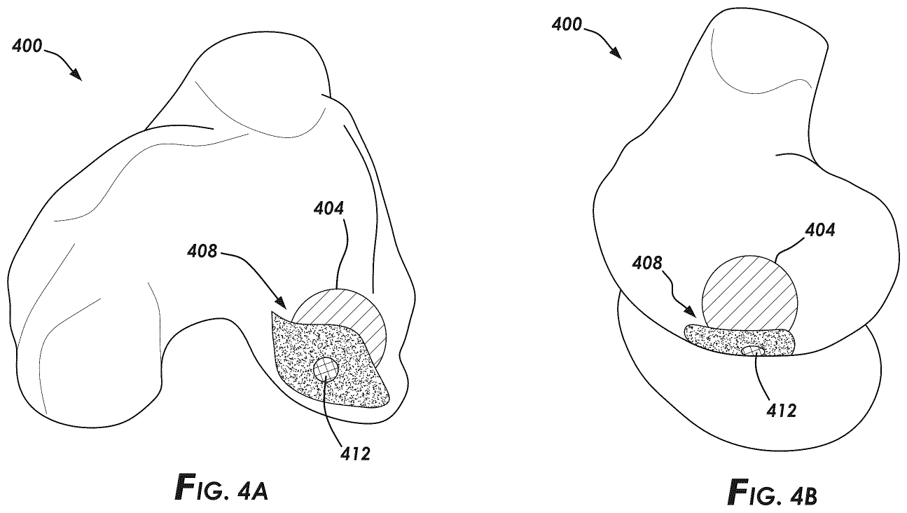

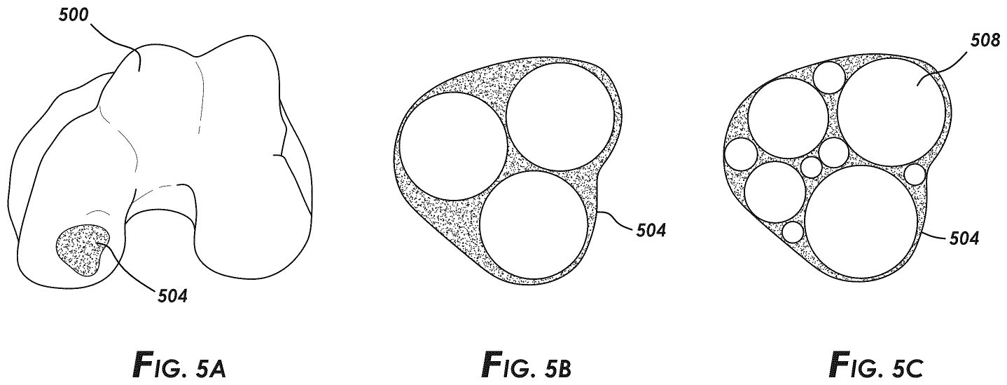

In some examples, a virtual plug generation is performed based on characteristics of the osteochondral defect (e.g., height, diameter, slope, etc.). FIGS. 5A, 5B, and 5C show an example distal femur 500 having a defect (e.g., in a distal medial femoral condyle) defined by an osteochondral boundary 504. An optimal pattern of plugs 508 within the defect site (e.g., as defined by the boundary 504) can be calculated based upon the result of the topographic matching as described above. As used herein, a pattern of plugs may refer to a plug assembly comprised of a plurality of plugs (e.g., an arrangement/or pattern of a plurality of different plugs configured to be arranged in the defect site). The 3D position and orientation of each graft/plug, as well as corresponding shape and other characteristics (e.g., diameter, height, and slope) can be selected to best rebuild the desired joint surface at the site of the defect. The pattern of plugs can be selected to cover as much of the defect as possible while minimizing the overlapping of plugs (since plug stability may be reduced by overlapping). The angle of graft placement (i.e., the angle between the osteochondral graft and the recipient site), the depth of graft harvest (which gives an indirect measurement of the height of the osteochondral graft harvested from a donor site), and graft placement (the depth to which the osteochondral graft is placed) can all be considered in the determination of plug selection and pattern.

For example, defects up to 9 mm in diameter may be filled using a single plug while larger defects may require with multiple plugs. In some examples, a 70 to 80% fill rate can be achieved with identical sized plugs. The use of variable-sized plugs (plugs having multiple radii) can increase the fill rate to between 90 and 100%. In this example, the plugs 508 over the defect site are placed perpendicular to the surface. However, in other examples, plug and pattern determination can include selecting slanted plugs and/or penalizing subsurface plug intersection (where a plug may be undercut by another plug, making the undercut plug more likely to loosen). Plugs of varying radius can be selected based upon the availability of suitable surgical tools. FIG. 5A shows an example pattern that includes three same-sized plugs (e.g., 10 mm diameter plugs). FIG. 5B shows an example pattern that includes two same-sized plugs (e.g., 8 mm diameter plugs) complemented with three varying-sized plugs (e.g., 2, 4 and 6 mm diameter plugs).

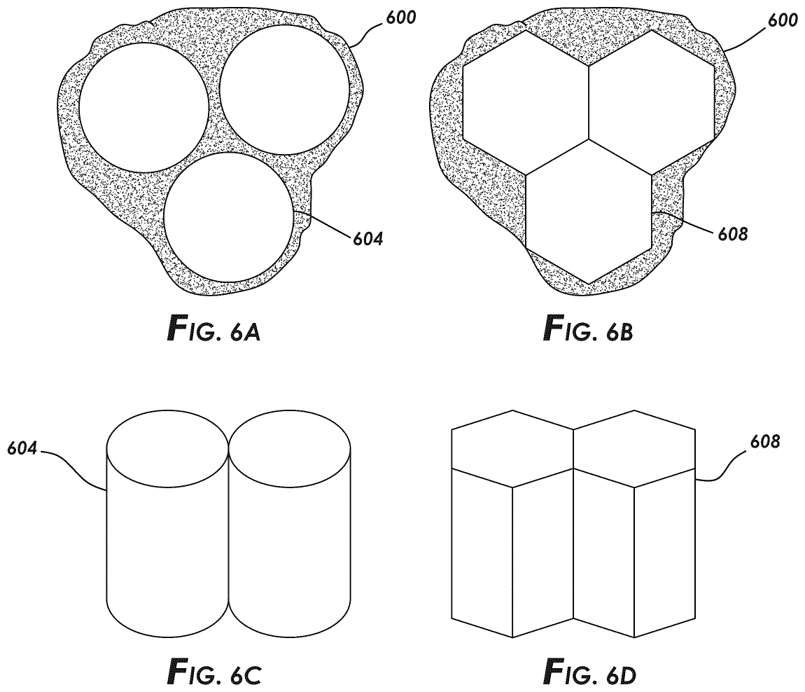

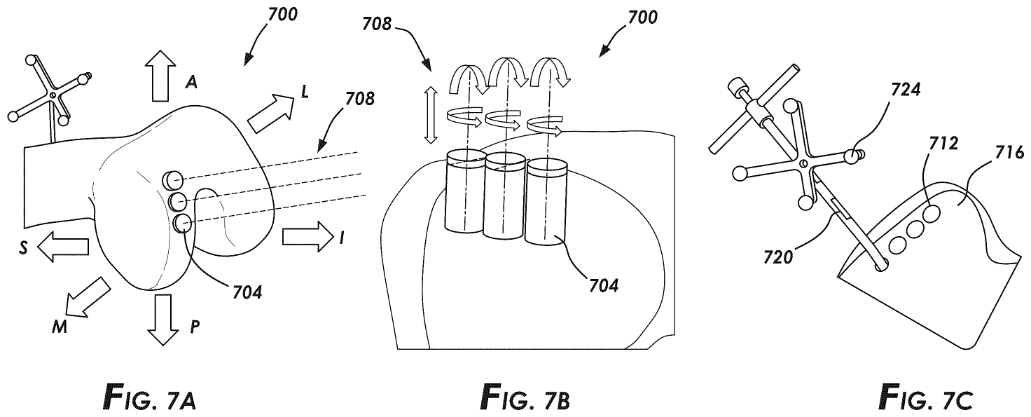

FIGS. 6A, 6B, 6C, and 6D show example plug patterns and shapes for a defect having an osteochondral boundary 600. FIG. 6A shows cylindrical grafts/plugs 604. Conversely, FIG. 6B shows hexagonal grafts/plugs 608. Although cylindrical grafts may used to cover defected area in mosaicplasty procedures, in some examples using cylindrical grafts may result in potential dead space left between the harvested grafts. Given that filling rate of the injured recipient osteochondral defect site can also play a role in long term clinical outcomes, harvesting grafts with hexagonal shape, which has the best volume geometry characteristics in nature, could be biomechanically advantageous and provide superior pull-out strength than cylindrical plugs. Further, it may be advantageous for a spongious bony bridge or wall of 1-2 mm in thickness to remain between graft holes in mosaicplasty. This spongious bone tissue between cylindrical grafts maintains a press-fit application of grafts, provides increased union, and enhances stability, especially in the short-term. However, it is not always possible to obtain this bony bridge with optimum thickness when cylindrical plugs are used. Conversely, contiguous positioning of hexagonal grafts provides full contact on sides of grafts without the need for a wall or bridge. For example, as shown in FIG. 6C, cylindrical grafts 604 can be arranged to have linear one-dimensional contact with one another. Conversely, as shown in FIG. 6D, hexagonal-shaped plugs 608 provide two-dimensional linear contact between the plugs solving the problem of gap spacing between traditional circular-shaped osteochondral plugs after transplantation and graft alignment using surgical navigation. These hexagonal “honeycomb” plugs, which are prism-shaped, are designed to press-fit together tightly, creating a more seamless and continuous surface after the procedure, demonstrating intact cartilage continuity. These plugs also support the transfer of grafts with solid tidemark and well-incorporated grafts leading to enhanced integration of bone and cartilage. Consequently, perioperative instability is reduced and alignment of the hexagonal plugs 608 is less challenging relative to the cylindrical plugs 604.

Further, the hexagonal grafts 608 may achieve 10-15% larger area coverage compared to the cylindrical plugs 604 since there is no gap between the adjacent grafts, and coverage of an even larger area may be possible. The option to use hexagonal graft techniques can be provided (e.g., via a software or other display interface prompt) as an alternative plug configuration in the planning stage, which could also include determining different sized tubes/complementary grafts to accommodate irregular-shaped defects in order to overcome any residual dead spaces after grafting. Another advantage of hexagonal grafts is that hexagonal grafts can be implanted into shallower defects relative to cylindrical grafts. The height and angulation of the harvested plugs can also controlled (e.g., by planning software) to avoid small amounts of recession or prominence in an autograft plug, which can lead to significant alterations in contact pressures at the grafted site.

A final surgical plan generated, stored, and/or output as described above may include a set of osteochondral grafts (“plugs”) positioned over the defect site, which may be visually displayed or otherwise provided to the surgeon. The 3D position and orientation of each plug, as well as plug shape (configuration, diameter, height, surface slope, etc.), can then be selected by the surgeon to best reconstruct the desired articular surface at the defect site.

Various techniques can be used to determine an optimal plug pattern or assembly. As one example, a shape-packing algorithm (e.g., a circle-packing algorithm, a hexagon-packing algorithm, etc.) can be used to determine the plug pattern.

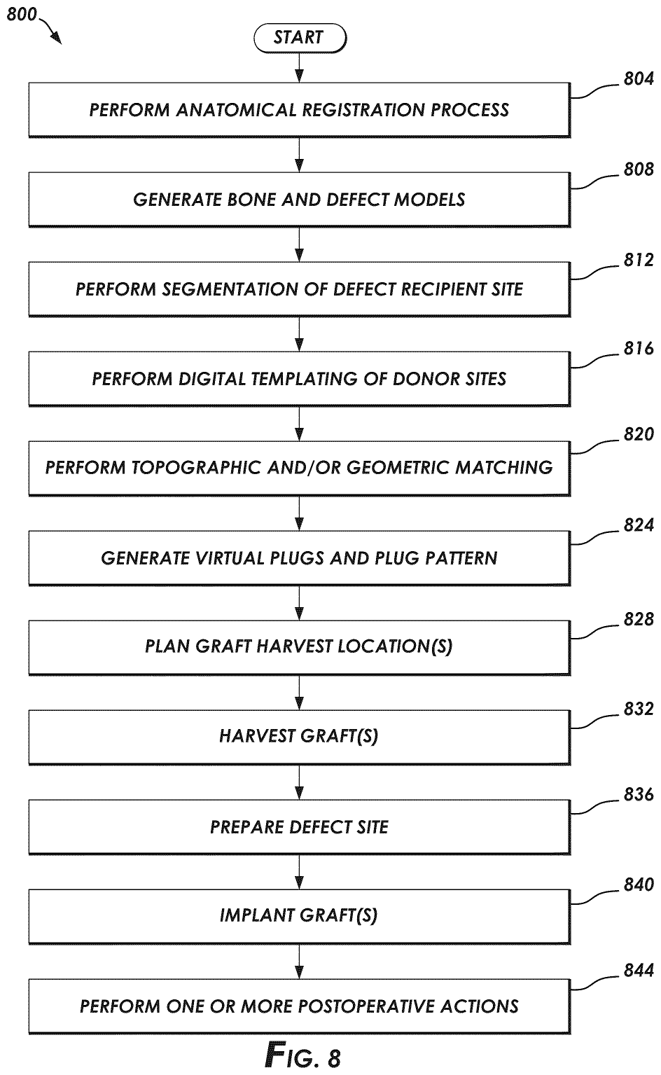

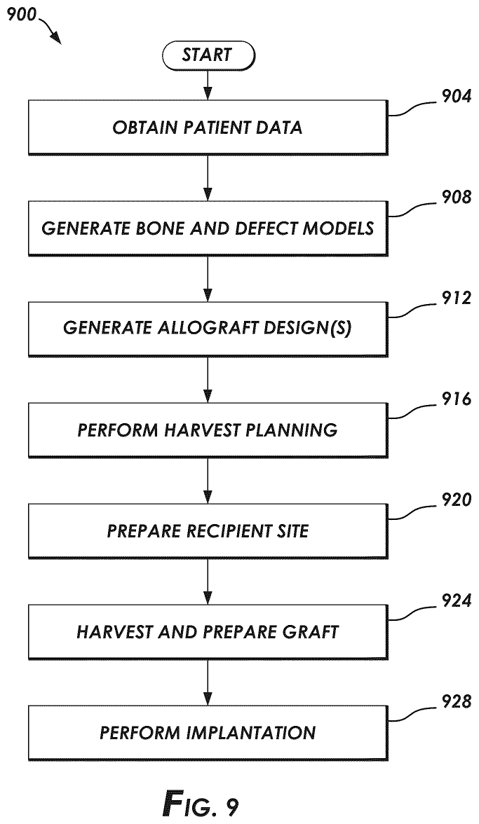

Referring now to FIGS. 7A, 7B, and 7C, once a pattern of plugs to fill the osteochondral defect has been provided, optimal locations from which to harvest the plugs can be determined in a graft harvesting location planning phase or stage to facilitate rebuilding the osteochondral defect. Graft harvesting planning techniques according to the present disclosure provide visualization of potential graft tunnel/plug sites (e.g., superimposed onto a 3D model of a femur 700 as shown in FIGS. 7A and 7B) recommended by the topographic matching algorithms/techniques described above. For each graft 704, a donor site can be selected to ensure the best fit with the shape of the graft. The grafts 704 can be rotated axially or translated along the plug axis so that the inclined surface of the harvesting site is best matched to the inclined surface of the defect site to obtain the best congruence of the joint surface. Techniques described herein provide measured deviations from the 90° position to the articular surface in six degrees of freedom (6DOF). For example, FIGS. 7A and 7B may correspond to visual guidance (e.g., as provided on a display screen) indicating the verticality of the virtual graft removal as well as placement according to the articular joint surface measured using the tunnel alignment wireframe trajectory as a surrogate for guide-to-bone angle as shown at 708. Visual guidance directions, as shown in FIG. 7A, may include anterior A, posterior P, lateral L, medial M, superior S, and inferior I directions. Angle of graft harvesting at the donor site can be measured with respect to a perpendicular line at the joint surface.

A best-fitting plug may be selected based upon the measured RMS surface error and the location of the plug relative to the articular surface. The 3D position and orientation of each plug, as well as plug shape (e.g., diameter, height, and surface slope) can also be selected to best reconstruct the desired articular surface at the registered defect site. For each plug, a harvest location can be selected to best match the shape of the plug based on data about the defect site as obtained using the techniques described above (e.g., topographic matching techniques). As describe above, the result of planning techniques described with respect to FIGS. 7A and 7B includes generating, storing, and/or outputting data corresponding to virtual harvest and implant positions and orientations of a set of plugs, along with the plug dimensions.

Example graft harvesting techniques (e.g., for harvesting the grafts planned/determined as described in FIGS. 7A and 7B) are described with respect to FIG. 7C. Osteochondral grafts 712 (corresponding to the grafts 704 planned/selected as described above) are harvested from a donor site (e.g., of a femur 716). In an example, a harvester/guide 720 having attached tracking sensors 724 is used. As an example, the donor site corresponds to a non-weight bearing area of a knee site as suggested/calculated by planning software/techniques as described herein. As shown, the guide 720 is inserted perpendicular to the joint surface to extract the osteochondral grafts/plugs 712. An appropriate perpendicular graft orientation can be achieved using the same tracked harvester/guide 720 to facilitate visualization of the orientation of the grafting/harvesting tools in real-time. A customized reference tracker references the exact position in relation to the predefined articular surface. Consequently, the permanent position of the guide tip and depth relative to the articular surface can be visualized. And displayed.

The surgeon can also use a tracked pointing device to locate the planned harvest site of a plug relative to the articulating surface. The surgeon positions and orientates the harvesting chisel on the cartilage (e.g., in accordance with visual guidance provided by planning software) from the lesser weight-bearing zone of the articulating surface. The surgeon then drives the chisel into the cartilage and bone until the guidance display indicates that a desired/target depth is reached (e.g., 15 mm). In an example, the harvester containing the cylindrical osteochondral graft is then removed by rotating the T-handle sharply by 90° twice to score and dislodge the graft. Each graft is then harvested separately and stored in sterile saline solution.

The recipient defect site preparation (e.g., graft hold preparation) can be performed using a robotic milling tool that can be operated in different modes depending upon the requirements of the surgical procedure (e.g., speed, exposure, rogue, etc.). As an example, an osteochondral defect can be excised in exposure control using a burr, which is navigated using planned planes of resection. The burr can also be operated in rogue control to create a level of macroscopic rough surface topography required to increase contact area and friction, which can promote plug stability. In some examples, the defect site can further be prepared with anti-rotation clocking features to aid plug alignment and stability. For example, a step or shoulder may be integrated into the base of a socket at the defect site.

In a graft implantation step, each harvested graft is delivered into the defect/recipient site (e.g., informed by the planning software) at an angle of 90° to the articular surface, with support of an optically tracked delivery guide applying pressure. This angle can be calculated as an angle of 0° for the instrument's longitudinal axis. The recipient site includes a hole having a depth configured to receive the harvested plug.

Various-post operative actions may be performed subsequent to implantation (e.g., shape confirmation of articular surface reconstruction). For example, upon completion of implantation, congruency of the articular surface can be measured (e.g., with the point probe) to detect any difference between the surface created by the grafts and the surface of the femoral condyle surrounding the grafts. Registration of anatomic points is an integral step in navigation. Therefore, logging of the coordinates of the articular surface facilitates identification and quantification of articular incongruities. The navigation system can also visualize the normal vector (a line perpendicular to the articular surface) for the generated surface, which is important for accurate graft placement. Graft height can then be calculated from the coordinates registered for the surrounding articular surface. Positive or negative RMS error values can be used to reflect the congruity (protrusion or subsidence of the graft) to the surrounding cartilage surface. Final adjustments can be made with the planning software and point probe to ensure that the graft is flush with the surface.

As described with respect to the OAT procedure in FIGS. 2-7, osteochondral arthroplasty techniques according to the present disclosure replace manual templating with high-resolution digitization of the lesion boundary and provide improved topographical matching of donor plugs from medial or lateral edge of trochlea (e.g., by considering angulation, thickness of the donor cartilage, etc.). Complex and time-consuming harvesting, trimming, and implanting of donor tissue steps are significantly reduced, and graft impaction and seating depth of plugs are improved through precise rotational clocking of donor plugs to the implant site. Further, integrity of the donor plugs can be improved by the inclusion of additional holes in the base of the defect for secondary fixation (e.g. screws, lugs, etc.).

In some examples, systems and methods according to the present disclosure are configured to display a planning screen or interface. The planning screen can display various types of visual guidance to a surgeon, such as defect preparation, graft harvesting, guided graft placement, and congruency check guidance. For example, for defect preparation, the system can guide the surgeon in preparing the defect site, ensuring proper fit and depth for the transplanted grafts. For graft harvesting, an angle of graft harvesting at the donor site, measured with respect to a perpendicular line at the joint surface, can be displayed/provided. For guided graft placement, an angle of graft placement (the angle between the osteochondral graft and the recipient site, as the graft is placed) can be displayed/provided. For congruency check, depth of graft harvest (which gives an indirect measurement of the height of the osteochondral graft harvested from a donor site) and graft placement (the depth to which the osteochondral graft is placed; ideally, this graft should be flush) can be displayed/provided. Perpendicularity in both recipient-site coring and graft harvest is desirable to have the most congruent graft placement possible and hence optimal joint surface congruity.

FIG. 8 shows an example method 800 for performing osteochondral arthroplasty techniques for an OAT procedure in accordance with the principles of the present disclosure. As described, the method 800 may be performed by one or more processing devices or processors, computing devices, etc., such as one or more controllers, computing devices, processors or processing devices, etc. of the surgical system 100 and may include executing instructions stored in memory. The method 800 includes controlling, in accordance with the functions described below, functions or operation of various components of the system 100, such as control of tools or instruments, one or more robots, display screens or other interfaces, etc. One or more steps of the method 800 may be omitted in various examples, and/or may be performed in a different sequence than shown in FIG. 8. The steps may be performed sequentially or non-sequentially, two or more steps may be performed concurrently, etc.

At 804, the method 800 includes performing an anatomical registration process to register patient anatomy using one or more of the techniques described herein. In some examples, imageless registration may be performed (e.g., using a point or touch probe, visual markers, etc.). In other examples, registration may include obtaining images (e.g., real-time or near real-time images) of a surgical environment including patient anatomy and one or more visual markers. Obtaining the images may include obtaining images using an arthroscopic camera or other imaging device configured to provide an image feed. In some examples, prior to obtaining the images, an image scan of patient anatomy may be performed, such as by performing a pre-operative imaging scan (e.g., a CT scan), retrieving the stored image scan (as data) from memory, etc. In other examples, other imaging techniques may be used. The images obtained from the image feed may be aligned with a model that is generated based on the image scan to provide visual guidance as described herein. The obtained images may include a sparse or dense set of images of the surgical environment including the one or more visual markers (e.g., at least one visual marker).

At 808, the method 800 includes generation of 3D virtual bone and focal defect models (e.g., as described above in FIGS. 2A and 2B), which may include performing an estimation of an original articular surface to construct a healthy (i.e., defect-free) femur model, obtaining or calculating characteristics of the defect, and calculating/digitizing a boundary of the defect. Generating the models or other digital data in step 808 may include and/or be referred to as generating first characteristics of the recipient or defect site.

At 812, the method 800 includes performing segmentation of an osteochondral defect recipient site. Segmenting the recipient site may include identifying (e.g., in an image or model of the recipient site) and segmenting the site into zones/sites such as a distal medial femoral condyle, a distal lateral femoral condyle, a posterior medial femoral condyle, a posterior lateral femoral condyle, etc. as described with respect to FIG. 3A. In some examples, segmenting the recipient site includes using image analysis or other techniques to identify and segment features of an image.

At 816, the method 800 includes performing digital templating of donor sites. Digital templating may include identifying, labelling, etc. (e.g., using image analysis techniques) zones, sub-zones or sites, etc. of donor sites as described with respect to FIG. 3B. Digital templating (or otherwise obtaining digital data associated with a donor site) in step 808 may include and/or be referred to as generating second characteristics of a donor site.

At 820, the method 800 includes performing topographic and/or geometric matching to obtain anatomic shape and curvature of both donor and recipient sites. As one example, matching may include comparing (i.e., comparing data indicative of) the relative curvature of any selected donor and recipient site on the articular surface of the distal femur. As another example, matching may include generating a best fit sphere as described above with respect to FIGS. 4A and 4B. Performing topographic and/or geometric matching may generally include comparing the first and second characteristics.

At 824, the method 800 includes generating one or more virtual plugs, assemblies, and/or patterns based on characteristics of the osteochondral defect (e.g., height, diameter, slope, etc.) as described above with respect to FIGS. 5A, 5B, 5C, 6A, 6B, and 6C. As one example, generating the plugs and patterns may include executing a best-fit or other optimization algorithm to determine, topographic/geometric characteristics of the defect site and the defect boundary, 3D positions and orientations, shapes, characteristics, etc. of each graft/plug. In some examples, plugs and patterns may be selected automatically (e.g., based on a coverage area achieved). In other examples, one or more plug shapes, patterns, configurations, etc. may be provided (e.g., via a display) to the surgeon for selection. Generating the one or more virtual plugs may include, generally, generating the one or more virtual plugs based on the comparison between the first characteristics and the second characteristics. In this manner, one or more virtual plugs that conform to the recipient site (e.g., fill the defect to optimize a fill percentage, fit without projecting above or being countersunk relative the surface surrounding the defect, etc.) are generated.

At 828, the method includes planning one or more graft harvest locations based on the plugs/grafts generated as selected at 824 (as described above with respect to FIGS. 7A, 7B, and 7C). Planning harvest locations may include providing (e.g., via a display) visualization of potential graft tunnel/plug sites. In some examples, donor sites can be selected automatically (e.g., based on a best fit in accordance with the grafts/plugs planned at 824). In other examples, harvest locations may be provided (e.g., via a display) to the surgeon for selection.

At 832, the method 800 includes harvesting one or more grafts/plugs. Grafts can be harvested by a surgeon (“manually”), using one or more robots (“robotically”), or combinations thereof. At 836, the method 800 incudes preparing the defect site to accept/receive the grafts. Preparing the defect site may include preparing one or more holes (e.g., shape, orientation, depth, etc.) based on shapes/characteristics of corresponding grafts. At 840, the method 800 includes implanting the grafts at the defect site (e.g., manually, robotically, or combinations thereof).

At 844, the method 800 includes performing one or more post-operative actions subsequent to implantation, such as measuring congruency of the articular surface and making adjustments.

Although described above with respect to OAT procedures, the principles of the present disclosure described with respect to FIGS. 2-8 can also be implemented for OCA procedures. In some examples, computer navigation software can be used to 3D render both the patient knee and allograft tissue and align the ideal articular surface angles and incision margins of the allograft to that of the patient's knee, including the optional screw insertions for graft stabilization. A femur tracker is then fixated into the diaphysis of the distal femur allograft. The allograft tissue can then be digitally scanned either intra-operatively using a point probe, which does not require cartilage segmentation to carry out the best fit between patient and donor, or pre-operatively using CT/MRI. Pre-operative CT/MRI segmentation, reconstruction, and the design of the allograft can be achieved using segmentation software. Reference points on both allograft and patient femur are also highlighted virtually that can be easily identifiable on both imaging and the patient anatomy, and also reachable with a probe tip during surgery. Surgical excision margins are also planned for the allograft and defect as well as mapping screw fixation insertion locations in optimal trajectories. Machining trajectories programming take into account the size of the recipient defect, the level of precision required to press-fit the allograft into the defect and the level of macroscopic rough surface topography required to increase contact area and friction, which can promote allograft stability.

The size of an irregular-shaped osteochondral defect can be established by demarcating the boundary with a point probe. The software then outputs a “best-fit” cylindrical shaped allograft. The osteochondral defect is excised using a burr, which is navigated using the planned planes of resection. As one example, a solid tungsten carbide fluted ball nose and shank cutter can be used as a bone cutting tool. A donor osteochondral allograft block is harvested using navigated cuts according to either pre-operative or intra-operative planning planes of resection to ensure the donor allograft matches as closely as possible to its recipient site in terms of size and articular congruency. In particular, burring of the deep surface of the graft to fit the cavity depth ensuring articular surface congruence. In an example, a robotic handpiece having a 6 degrees of freedom (DoF) milling machine can be programmed to machine allograft tissue to a specific shape and size based upon the tool path trajectories created during the planning stage. This is required for complex shapes with significant undercuts or elaborate geometries. Specifically, there are three axes are for spatial translation of the tool, while the other three axes are used for orientation. The osteochondral allograft is then inserted into the patient's debrided defect and stabilized with screws or pins guided by the navigation software.

The example OAC techniques as described above provide the ability to create singular non-circular shapes and reduce perilesional excision margins, match bone and cartilage depths of allograft to host tissue, and match allograft bone surface curvature to the surrounding host. Further, these techniques create smaller singular allografts to fit irregular defect shapes, minimizing impaction and avoiding many of the issues associated with larger allograft size, and provide the ability to perform geometric and topographic matching of non-medial femoral condyle allografts to medial condyle osteochondral defects. In some examples, registration error with allograft and patient femur alignment in the peri-operative phase is reduced. These techniques are applicable to allograft selection and implantation for tumors, complex fracture non-union or mal-union management, and correction of skeletal deformities to improve size/shape matching.

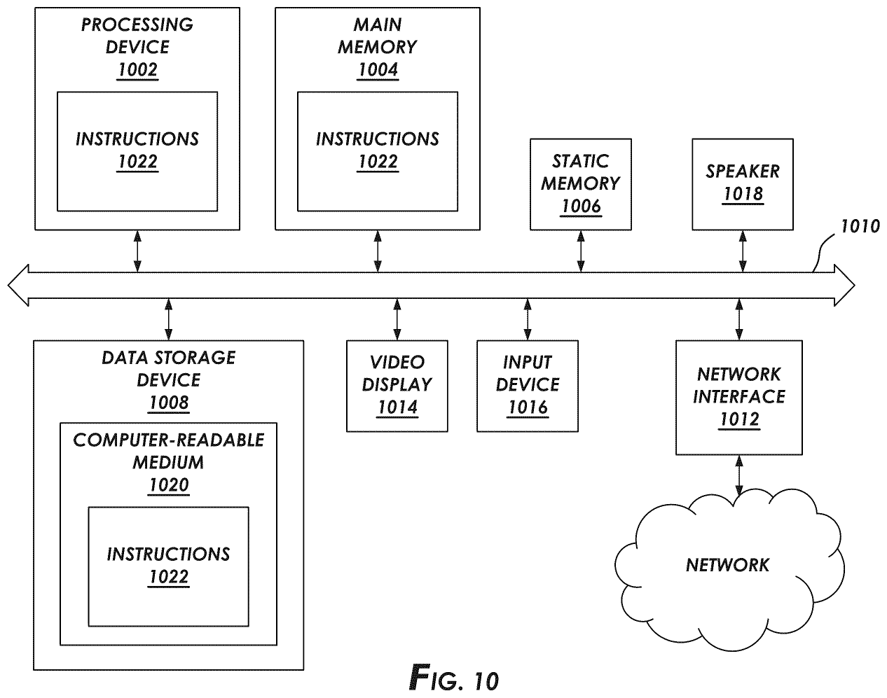

FIG. 9 shows an example method 900 for performing osteochondral arthroplasty techniques for an OCA procedure (e.g., a robot-assisted allograft procedure) in accordance with the principles of the present disclosure. As described, the method 900 may be performed by one or more processing devices or processors, computing devices, etc., such as one or more controllers, computing devices, processors or processing devices, etc. of the surgical system 100 and may include executing instructions stored in memory. The method 900 includes controlling, in accordance with the functions described below, functions or operation of various components of the system 100, such as control of tools or instruments, one or more robots, display screens or other interfaces, etc. One or more steps of the method 900 may be omitted in various examples, and/or may be performed in a different sequence than shown in FIG. 9. The steps may be performed sequentially or non-sequentially, two or more steps may be performed concurrently, etc.

At 904, the method 900 includes obtaining data corresponding to patient anatomy, which may include obtaining patient CT data. In some examples, an anatomical registration process may be performed to register patient anatomy using one or more of the techniques described herein. In some examples, imageless registration may be performed (e.g., using a point or touch probe, visual markers, etc.). In other examples, registration may include obtaining images (e.g., real-time or near real-time images) of a surgical environment including patient anatomy and one or more visual markers. Obtaining the images may include obtaining images using an arthroscopic camera or other imaging device configured to provide an image feed. In some examples, prior to obtaining the images, an image scan of patient anatomy may be performed, such as by performing a pre-operative imaging scan (e.g., a CT scan), retrieving the stored image scan (as data) from memory, etc. In other examples, other imaging techniques may be used. The images obtained from the image feed may be aligned with a model that is generated based on the image scan to provide visual guidance as described herein. The obtained images may include a sparse or dense set of images of the surgical environment including the one or more visual markers (e.g., at least one visual marker).

At 908, the method 900 includes generation of various models or other data (e.g., using the data obtained at 904, by performing various scans or other image processing techniques, using registration techniques, etc.) associated with the recipient site and/or the donor site. The models may include 3D virtual bone and defect models corresponding to patient anatomy as well as one or models of an allograft, an allograft donor site, etc. Generating the models may include performing a point cloud collection process for geometric data acquisition and performing the modelling based on the acquired data.

As one example, the osteochondral defect can be identified and digitized as described herein, such as by identifying a contour, demarcating the boundary (e.g., using a point probe), etc. As another example, a femur tracker can be fixed into the diaphysis of the distal femur allograft. The allograft tissue can then be digitally scanned (either intra-operatively using a point probe or a miniature laser scanner or RGB-D camera, which does not require cartilage segmentation to carry out the best fit between patient and donor, or pre-operatively using CT/MRI).

At 912, the method 800 includes generating an allograft design (or designs) based on the models. For example, generating the allograft design may include performing one or more segmentation, reconstruction, and design steps, such as by using 3D slicer software. Performing segmentation of an osteochondral defect recipient site may include identifying (e.g., in an image or model of the recipient site, based in part on the identified defect) and segmenting the site into zones/sites as described with respect to FIG. 3A. In some examples, segmenting the recipient site includes using image analysis or other techniques to identify and segment features of an image. One or more “best-fit” allografts can then be generated based on characteristics of the recipient and donor sites identified defect.

At 916, the method 900 includes performing one or more harvest planning steps. Harvest planning may include, but is not limited to, generating alignment data indicating alignment between the ideal articular surface angles and incision margins of the allograft to that of the patient's knee, including optional screw or anchor insertions for enhanced graft stabilization. Reference points on both the allograft (donor) site and patient femur can also be highlighted virtually to be easily identifiable on both imaging and the patient anatomy, and also reachable with a probe tip during surgery. Surgical excision margins can also be planned for the allograft and defect as well as mapping screw fixation insertion locations in optimal trajectories. Machining trajectories programming take into account the size of the recipient defect, the level of precision required to press-fit the allograft into the defect, and the level of macroscopic rough surface topography required to increase contact area and friction, which can promote allograft stability.

At 920, the method 900 includes preparing the recipient site. Preparing the recipient site may include, for example, milling/excising the defect (e.g., using a burr or other instrument) based on characteristics of the allograft(s) design.

At 924, the method 900 includes harvesting and preparing the graft. Harvesting and preparing the graft may include harvesting the allograft in accordance with the allograft design and machining the allograft to obtain optimal fit at the defect site.

At 928, the method 900 includes performing implantation of the allograft into the prepared recipient site. In some examples, implantation includes stabilizing the allograft using screws, pins, etc.

FIG. 10 shows an example computer system or computing device 1000 configured to implement the various systems and methods of the present disclosure. In one example, the computer system 1000 may correspond to one or more computing devices of the system 100, a tablet device within the surgical room, or any other system that implements any or all the various methods discussed in this specification. For example, the computer system 1000 may be configured to implement all or portions of the methods 800 and 900. The computer system 1000 may be connected (e.g., networked) to other computer systems in a local-area network (LAN), an intranet, and/or an extranet (e.g., a network associated with the system 100 and/or device cart 102), or at certain times the Internet (e.g., when not in use in a surgical procedure). The computer system 1000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1000 includes a processing device 1002, a main memory 1004 (e.g., read-only memory (ROM), flash memory, dynamic random access memory (DRAM) such as synchronous DRAM (SDRAM)), a static memory 1006 (e.g., flash memory, static random access memory (SRAM)), and a data storage device 1008, which communicate with each other via a bus 1010.

Processing device 1002 represents one or more general-purpose processing devices such as a microprocessor, central processing unit, or the like. More particularly, the processing device 1002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1002 may also be one or more special-purpose processing devices such as an application specific integrated circuit (ASIC), a field programmable gate array (FPGA), a digital signal processor (DSP), network processor, or the like. The processing device 1002 is configured to execute instructions for performing any of the operations and steps discussed herein. Once programmed with specific instructions, the processing device 1002, and thus the entire computer system 1000, becomes a special-purpose device, such as one or more of the controllers of the surgical system 100.

The computer system 1000 may further include a network interface device 1012 for communicating with any suitable network (e.g., the device cart 102 network). The computer system 1000 also may include a video display 1014 (e.g., the display device 114), one or more input devices 1016 (e.g., a microphone, a keyboard, and/or a mouse), and one or more speakers 1018. In one illustrative example, the video display 1014 and the input device(s) 1016 may be combined into a single component or device (e.g., an LCD touch screen).

The data storage device 1008 may include a computer-readable storage medium 1020 on which the instructions 1022 (e.g., implementing any methods and any functions performed by any device and/or component depicted described herein) embodying any one or more of the methodologies or functions described herein is stored. The instructions 1022 may also reside, completely or at least partially, within the main memory 1004 and/or within the processing device 1002 during execution thereof by the computer system 1000. As such, the main memory 1004 and the processing device 1002 also constitute computer-readable media. In certain cases, the instructions 1022 may further be transmitted or received over a network via the network interface device 1012.

While the computer-readable storage medium 1020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

What is claimed is:

1. A system for performing an osteochondral grafting procedure, the system comprising:
memory storing instructions; and
one or more processing devices configured to execute the instructions, wherein executing the instructions causes the one or more processing devices to
obtain first digital data of an anatomical site of a patient, wherein the anatomical site includes a recipient site for an osteochondral graft, and wherein the recipient site includes an osteochondral defect,
determine, based on the first digital data, first characteristics of the recipient site,
obtain second digital data of a donor site,
determine, based on the second digital data, second characteristics of the donor site, and
generate, based on the first characteristics and the second characteristics, at least one virtual plug corresponding to the osteochondral graft, wherein the at least one virtual plug is configured to conform to the recipient site, and wherein generating the at least one virtual plug includes at least one of (i) storing data defining the at least one virtual plug and (ii) providing, on a display, visual guidance based on the at least one virtual plug.

2. The system of claim 1, wherein the osteochondral grafting procedure includes at least one of an osteochondral autograft transplantation (OAT) procedure and an osteochondral allograft (OCA) procedure.

3. The system of claim 1, wherein:
obtaining the first digital data includes generating a surface model of the anatomical site and performing segmentation of the recipient site based on the surface model;
obtaining the second digital data includes obtaining a digital template of the donor site; and
generating the at least one virtual plug includes performing a matching process to obtain anatomic shapes and curvatures of both the recipient site and the donor site and generating the at least one virtual plug based on results of the matching process.

4. The system of claim 3, wherein obtaining the digital template includes digitally segmenting the donor site into a plurality of donor sites.

5. The system of claim 4, wherein performing the matching process includes comparing shapes and curvatures of the recipient site to shapes and curvatures of the plurality of donor sites.

6. The system of claim 4, wherein performing the matching process includes generating the at least one virtual plug based on one of the plurality of donor sites and superimposing the at least one virtual plug on the recipient site.

7. The system of claim 1, wherein generating the at least one virtual plug includes determining a topographical match between the recipient site and the donor site based on the first and second characteristics.

8. The system of claim 1, wherein generating the at least one virtual plug includes at least one of translating and rotating the at least one virtual plug to assess conformity of the at least one virtual plug to the recipient site.

9. The system of claim 8, wherein assessing the conformity includes calculating an average error of a topographic mismatch between the at least one virtual plug and the recipient site.

10. The system of claim 8, wherein assessing the conformity includes calculating a least squares distance error between respective surfaces of the at least one virtual plug and the recipient site.

11. The system of claim 1, wherein the first characteristics include first radius of curvature data for the recipient site and the second characteristics include second radius of curvature data for the donor site, and wherein generating the at least one virtual plug includes generating a best-fit sphere based on the first radius of curvature data and the second radius of curvature data.

12. The system of claim 1, wherein generating the at least one virtual plug includes identifying respective diameters, heights, and surface slopes of a plurality of virtual plugs based on the first and second characteristics.

13. The system of claim 1, wherein generating the at least one virtual plug includes executing a shape-packing algorithm to select a plurality of virtual plugs.

14. The system of claim 1, wherein executing the instructions further causes the one or more processing devices to control a robot to at least one of (i) obtain, based on the at least one virtual plug, the osteochondral graft from the donor site and (ii) implant the osteochondral graft at the recipient site.

15. A method for performing an osteochondral grafting procedure, the method comprising, using one or more processing devices:
obtaining first digital data of an anatomical site of a patient, wherein the anatomical site includes a recipient site for an osteochondral graft, and wherein the recipient site includes an osteochondral defect;
determining, based on the first digital data, first characteristics of the recipient site;
obtaining second digital data of a donor site;
determining, based on the second digital data, second characteristics of the donor site; and
generating, based on the first characteristics and the second characteristics, at least one virtual plug corresponding to the osteochondral graft, wherein the at least one virtual plug is configured to conform to the recipient site, and wherein generating the at least one virtual plug includes at least one of (i) storing data defining the at least one virtual plug and (ii) providing, on a display, visual guidance based on the at least one virtual plug.

16. The method of claim 15, wherein the osteochondral grafting procedure includes at least one of an osteochondral autograft transplantation (OAT) procedure and an osteochondral allograft (OCA) procedure.

17. The method of claim 15, wherein:
obtaining the first digital data includes generating a surface model of the anatomical site and performing segmentation of the recipient site based on the surface model;
obtaining the second digital data includes obtaining a digital template of the donor site; and
generating the at least one virtual plug includes performing a matching process to obtain anatomic shapes and curvatures of both the recipient site and the donor site and generating the at least one virtual plug based on results of the matching process.

18. The method of claim 15, wherein generating the at least one virtual plug includes at least one of:
determining a topographical match between the recipient site and the donor site based on the first and second characteristics;
at least one of translating and rotating the at least one virtual plug to assess conformity of the at least one virtual plug to the recipient site;
identifying respective diameters, heights, and surface slopes of a plurality of virtual plugs based on the first and second characteristics; and
executing a shape-packing algorithm to select a plurality of virtual plugs.

19. The method of claim 15, wherein the first characteristics include first radius of curvature data for the recipient site and the second characteristics include second radius of curvature data for the donor site, and wherein generating the at least one virtual plug includes generating a best-fit sphere based on the first radius of curvature data and the second radius of curvature data.

20. The method of claim 15, further comprising controlling a robot to at least one of (i) obtain, based on the at least one virtual plug, the osteochondral graft from the donor site and (ii) implant the osteochondral graft at the recipient site.
